(19) United States
(12) Patent Application Publication
(10) Pub. No.: US 2007/0276417 A1
(43) Pub. Date: Nov. 29, 2007

(54) Laterally Curved Surgical Clip

(76) Inventors: Jose Barbosa Mendes Jr, Parana (BR); Milton Tatsuo Tanaka, Parana (BR)

Correspondence Address: RYNDAK & SURI LLP, 200 W. MADISON STREET, SUITE 2100, CHICAGO, IL, 60606, US

(21) Appl. No.: 10/594,186

(22) PCT Filed: Jun. 8, 2005
(86) PCT No.: PCT/BR05/00102

§ 371 Date: Sep. 25, 2006

(30) Foreign Application Priority Data
Jul. 2, 2004 (BR) MU 8.401.529-2

(51) Int. Cl. A61B 17/08

(52) U.S. Cl. 606/157

(57) ABSTRACT

This invention is a self-locking, surgical instrument designed to be utilized in surgical procedures where total and/or partial vascular occlusion of large blood vessels, or other structures, is needed, thus providing secure stanching of the vessels and structures due to its curved lateral design. This innovative design not only allows for a clear view of the entire extension of the clip, but also has a “male-to -female” self-locking system, which stays clear of the area being clipped, thus constituting an improvement upon the existing traditional straight clips.

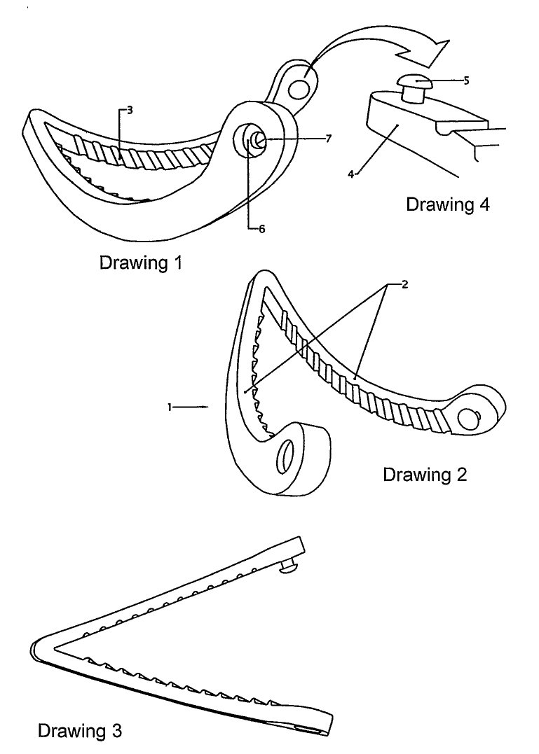

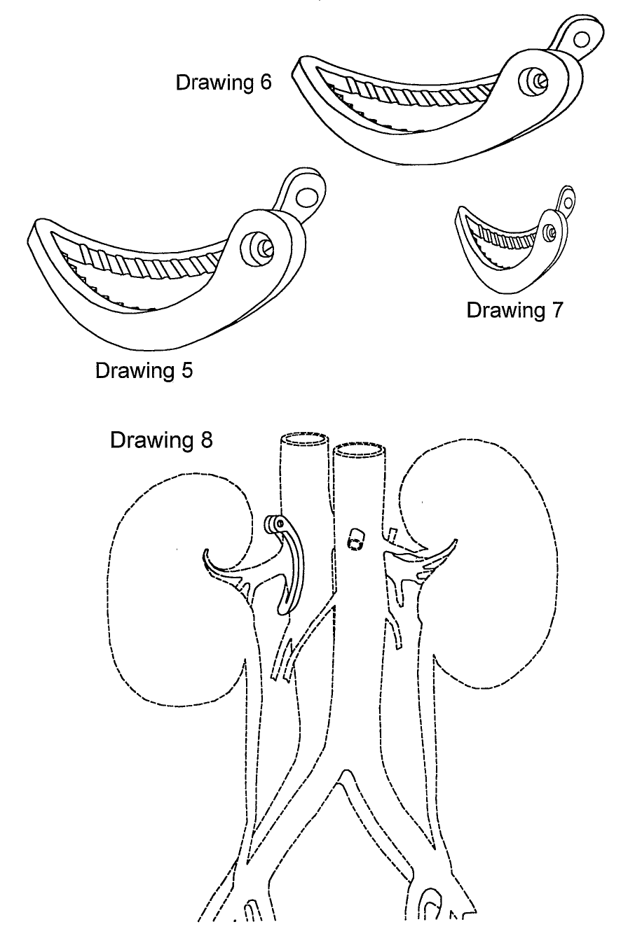

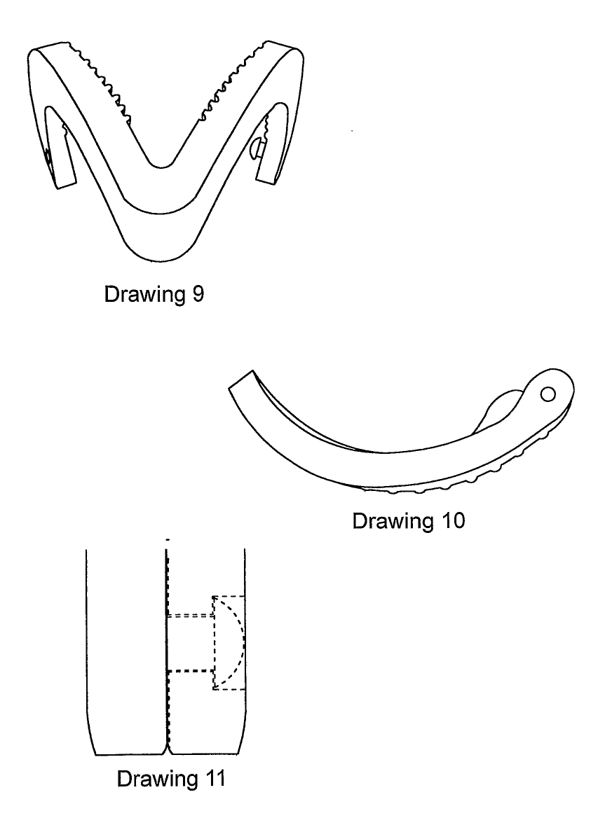

### PRESENTATION

This utility model patent report describes a Laterally Curved Laparoscopic Surgical Clip, which is a self-locking surgical instrument. It is used in surgeries where vascular occlusion, or the occlusion of other structures is needed. Since it has an innovative curved lateral design it not only provides for secure stanching of the vessels and structures but also has a self-locking male-to-female system, while allowing for the viewing of the entire clip extension making it possible to leave the locking mechanism away from the clipped area. Altogether, this makes up for great improvements when compared to the existing linear designed clips.

### INTRODUCTION

Laparoscopic surgery is a surgical modality which is well established in several surgical areas, from extirpative to reconstructive procedures. Due to the fact that it is a minimally invasive modality of surgery, it has been considered as the “gold standard” for some procedures. For that reason, laparoscopic surgery is now, as it will continue to be in the near future, the main approach to surgery.

From the surgical anatomical point of view, the organs that are more vascularized and having the pediculi with the largest calibers are the ones that offer the biggest technical challenges when being dealt with. That is why unexpected surgical problems during the operation are the most frequent causes of conversions to open surgery or even loss of patient.

Within this context, the urinary system occupies a high profile position, because we know that, at an average, every 5 minutes the entire blood volume passes through the kidneys. On top of this organ's perfusion characteristics, the anatomical variations of its pedicule (arterial and venous multiplicity, precocious arterial bifurcations, anomalous venous merging and others) are very frequent.

Generally, the nephrectomies, for many reasons, are one of the main urological procedures. The current modalities of available renal pedicule control are: classical ligatures with the use of threads, sealing by means of different electromedicine techniques (harmonic scalpel, thermal and electrocautery equipment, lasers and others), vascular clipping and stapling equipment. Due to the technical difficulties of the classical ligatures (thread), which demand more time for their realization, the use of clips have become one of the preferred forms of ligatures by most surgeons who consider practicality and speed. Despite its practicality and speed, the use of electronic equipment (electromedicine) faces some limitations when it comes to working with large vessels or when one considers the operational cost. In spite of its elevated cost, endostaplers have been developed with the intention of joining safety to speed and the practicality of the clips.

### TECHNICAL STATUS

The technical status of such equipment is innovative because it does not count on exclusive and specific instruments for its destined use, i.e., there is no knowledge of the existence of surgical clips with lateral curvature utilized along with laparoscopic surgeries or even of the open kind. Among the documents found during the state-of-the-art searches, it was possible to select the patent of a generic surgical clip with similar characteristics to our product. Thus, the following document has been picked out:

Patent number U.S. Pat. No. 5,062,846, which presents a clip for generic use, has a curvature only in the internal faces of the occlusion when closed, that is, it doesn't present any lateral curvature of the piece itself when closed.

### PROPOSED DEVELOPMENT

The surgical apparatus hereby presented is based on a technical, surgical fundament of the classic utilization of curvature in the majority of surgical instruments that are employed for the dissection of organs and vascular structures. This design has been conceived with the intent to incorporate these fundaments to the usage of clips and it constitutes an innovative improvement for their use and application. Therefore, it is the first laterally curved conceived device, self-locking, or not. Since it allows for lateral and partial applications on large vessels or other structures, the lateral curvature provides the clip with advantages over the straight ones. Due to its curved shape, this clip has two working surfaces: an effective one, which is meant to be used with vascular occlusion, and a non-effective one, which is used with the locking system, thus avoiding the clipped vascular structure. Another advantage of its curved shape is based on the surgeon's better visualization of the clip locking end, thus avoiding undesirable inclusions of tissues or perivascular structures. Therefore, since it is possible to view the entire length of the clip, it allows for secure sectioning of the vessel, i.e., the faces of the occlusion where the lateral grooves and protrusions are located are both coplanar or flat in relation to each other, although curved in relation to its major view axis (front), thus allowing for partial capturing of the wall in a large vessel. It leaves its locking portion away from the structure being worked on, and doesn't perforate, tear and/or cause any lesion to it.

The locking system on this curved clip is based on a “male-to-female” type, and is incorporated in the clip's free end, where a mushroom-shaped pin can, under pressure, be adjusted to an orifice. Both the “male” and “female” parts are placed in the central part of the free end. Once it is locked in place, this clip does not present any risk of spontaneous or accidental opening. This locking system is designed to facilitate the manufacturing and the operation of the device. In order to lock it, pressure is exerted along one direction only of the applicator, thus eliminating the need to displace one leg over the other in order to lock it in place. Moreover, such applicator should be able to rotate 360° around its distal end, in order to allow for the same curved clip to be applied both to the left and to the right.

In order to provide this device with a lower risk of slippage from the clipped vessel wall, there are grooves and transversal protrusions that self adjust, therefore providing the device with an additional locking mechanism for the structures.

There is a technical variant of this system, which is incorporated along the occlusion face of the clip and is based on the longitudinal disposition of grooves along one of the legs and protrusions along the opposite leg. These also self adjust, and the purpose is again to provide a better grip of the structures.

As far as the confection material is concerned, the lateral curvature principle of a clip can be applied in polymers, as well as in metal, depending on the application. When using polymers for the confection of the clip, the locking mechanism should be present, however, with the metallic clip there's no need for a locking mechanism since the deformation of the clip itself will provide the needed pressure for the occlusion.

The Laterally Curved Laparoscopic Surgical Clip may present variations in size and the curvature angles, depending on the diameter of the vessels or the application which it is destined to be used on. This does not mean that the original principle should be altered.

### EXAMPLES OF APPLICATIONS

Within urology, for example, the anatomy of kidneys presents the following particularities:

The right kidney vein is rather short, and since the vena cava is located at the right of the aorta, the nephrectomy of a live donor for transplantation is preferably done at the left side, thus enabling the extraction of a venous segment which is long enough to be worked on. The importance of the length of this vein is that the venous reconstruction is done through a terminal-lateral anastomosis, thus, if we are working with a short vein segment this anastomosis will offer some technical difficulties as well as complication risks due to obstruction by thrombosis. This happens due to an imperfect suture or to stretching of anastomosed vessels. Considering that the surgery of the donor presents unique characteristics when it comes to surgical risks, for it deals with an individual who does not present any pathology and is being submitted to a major surgery, the standard technical modality for vein control has been the usage of vascular staplers. However, due to fact that they have three rows of staples along each side, a loss of one of the vein segments of about 1.5 centimeters takes place. Because of the factor herein pointed out, the utilization of the right kidney for transplantations has been very limited, even in anatomical variations for the left side of the double artery type, which infers the need for bench surgery, which translates into a longer ischemia of the implant, as well as a higher risk of vascular complications. With the advent of the product herein presented, it is both possible and safe to have an application of a device which partially includes the wall of the vena cava, therefore allowing for the removal of a longer segment of the kidney vein without any damage attributed to the use of vascular staplers and with no risk increase to the donor.

Another application where this clip would excel is on unexpected vascular problems, where there are lesions in the walls of the large vessels that cannot be totally connected or cauterized. Therefore, in this situation, the only possibility would be the classical vascular sutures or hypothetically the partial clipping of the lesioned vessel, which could be performed in a secure and effective way, but up till now there isn't a clip that makes such repair possible. Beyond the advantages herein mentioned, we also have to consider the previously mentioned visualization factor of the entire length of the clip, which provides more safety when clipping and locking it on the vessel. With all of these technical and safety advantages, the utilization of such curved clips, even in pathological nephrectomies, either benign or malignant in nature, would be justified, thus increasing the possibilities of usage.

Due to its enormous advantages when compared to other kinds of clips available in the market, we cannot limit the specific usage of the curved surgical clip to urologic surgeries. We should include its utilization in other non-urologic procedures, such as: splenectomy, partial hepatectomy or pneumectomy (and in these cases, its curvature would be extremely useful for clipping the sectioned biliary ducts or bronchioles respectively), and small lesions in the bowels, among others.

### DETAILED DESCRIPTION OF THE PRODUCT

The Laterally Curved Laparoscopic Surgical Clip is composed of a structure of two convex legs, therefore presenting a slim and innovative curved design containing an array of internal transversal ‘teeth’ (3) which can also be presented longitudinally along the legs, thus contributing for the occlusion and locking of vessels, or other structures.

The locking system granted to this curved clip is placed at the terminal part of the device (4) and is based on a “male-to-female” system where a mushroom-like “male” pin fits, under pressure, into a “female” orifice. Both are placed in the center of the free extremity and once locked there is no risk of spontaneous opening.

The Laterally Curved Laparoscopic Surgical Clip has other configuration variants, as presented in drawings 5, 6 and 7. These differences are related to size, and angulation of the curvatures.

### DESCRIPTIONS OF THE DRAWINGS

What follows is a series of illustrations which represent the Laterally Curved Laparoscopic Surgical Clip.

These drawings are merely illustrative and the final product may present some variations which shall not deviate from the original idea behind the product.

They are as follows:

Drawing 1: A perspective view of the proposed object. This view emphasizes the lateral curves of both legs, as well as the locking mechanism.

Drawing 2: Another perspective view of the object which highlights the curvature and the internal “teeth” along the right and left legs.

Drawing 3: An orthogonal top view of the surgical clip shown in drawing 1.

Drawing 4: A detailed magnified perspective view, highlighting the mushroom-like head of the male to female locking system.

Drawing 5: A perspective view of a configuration variant of the clip shown in drawing 1.

Drawing 6: A perspective view of a second configuration variant of the clip shown in drawing 1.

Drawing 7: A perspective view of a third configuration variant of the clip shown in drawing 1.

Drawing 8: A perspective view of the surgical clip, emphasizing the front view, which shows the positioning of the clip when applied to a vessel during a laparoscopic surgery—in this case including a partial apprehension of the vena cava during a right nephrectomy.

Drawing 9: A perspective view which shows the curvature of the proposed object and highlights the front and top views, as well as the locking system.

Drawing 10: An orthogonal side view of the clip.

Drawing 11: A detailed magnified perspective view of the locking mechanism of the clip.

1-4. (canceled)

5. A surgical clip comprising:
a pair of laterally curved legs;
a flexible articulation joining the pair of legs at one end; and
a locking mechanism at a second end of the pair of legs for securing the legs together in a closed position.

6. The surgical clip of claim 5 wherein the locking mechanism comprises a pin located at the free end of one leg and an orifice located at the free end of the other leg, the pin being adapted to snap fit under pressure into the orifice.

7. The surgical clip of claim 5 wherein each leg includes a plurality of teeth on its inner surface.

8. The surgical clip of claim 7 wherein the teeth are transversely oriented on each leg.

9. The surgical clip of claim 7 wherein the teeth are longitudinally oriented on each leg.

10. The surgical clip of claim 5 wherein the clip is formed of metal.

11. The surgical clip of claim 5 wherein the clip is formed of polymer material.

12. The surgical clip of claim 5 wherein the flexible articulation is integral with the legs.

13. A surgical clip comprising:
a pair of laterally arcuate legs;
a flexible articulation joining the pair of legs at one end; and
a locking mechanism at a second end of the pair of legs for securing the legs together in a closed position;
wherein one leg includes a longitudinal ridge on its inner surface and the other leg includes a longitudinal recess on its inner surface, the ridge being adapted to fit within the recess when the clip is in the closed position.

14. The surgical clip of claim 13 wherein the locking mechanism comprises a pin located at a central portion of the free end of one leg and an orifice located on a central portion of the free end of the other leg, the pin being adapted to snap fit under pressure into the orifice.

15. The surgical clip of claim 13 wherein the flexible articulation is integral with the legs.

16. A surgical clip for occluding a vessel, comprising:
A pair of laterally curved legs, each leg having an inner surface and an outer surface, the inner surfaces of the legs being parallel and in contact with one another when the clip is in a closed position, one leg having at least one protrusion on its inner surface and the other leg having at least one recess on its inner surface, the protrusion and recess being adapted to fit together when the clip is in a closed position;
A flexible articulation joining the pair of legs at one end; and
A locking mechanism at a free end of the pair of legs for holding the legs together in a closed position.

17. The surgical clip of claim 16 wherein the locking mechanism comprises a pin located at a central portion of the free end of one leg and an orifice located on a central portion of the free end of the other leg, the pin being adapted to snap fit under pressure into the orifice.

18. The surgical clip of claim 16 wherein the flexible articulation is integral with the legs.

19. A metal surgical clip comprising:
a pair of laterally curved legs, each leg having an inner surface and an outer surface; and
a flexible articulation joining the pair of legs at one end;
wherein the clip has an open position and a closed position, the clip being lockable in the closed position by deformation of the clip from the open position by applying sufficient force to press the inner surfaces of the legs together.

20. The surgical clip of claim 19 wherein each leg includes a plurality of teeth on its inner surface.

21. The surgical clip of claim 20 wherein the teeth are transversely oriented on the inner surface of each leg.

22. The surgical clip of claim 20 wherein the teeth are longitudinally oriented on the inner surface of each leg.

23. The surgical clip of claim 19 wherein the flexible articulation is integral with the legs.

24. The surgical clip of claim 19 wherein one leg includes a longitudinal ridge on its inner surface and the other leg includes a longitudinal recess on its inner surface, the ridge being adapted to fit within the recess when the clip is in the closed position.
